SEQUENCE LISTING (9 sequences)

SEQ ID NO: 1 - DNA, 830 bp, mouse
ccaggatcaagagtcaccgcttcgcaagcactgcctggctccatcaggatccccgcaggctcagctccaaggcaccgctcaccaggaaggcatcatgggcttcctgaagttctcccctttcctggttgtcagcatcttgctcctgtaccaggcatgcagcctccaggcagtgcctttgaggtcaatcttggaaagcagcccaggcatggccactctcagtgaagaagaagttcgcctgctggctgcactggtgcaggactatatgcagatgaaacccagggagctggagcaggaggaagagcaggaggctgagggctctagcttggacagccccagatctaagcggtgtgggaatctgactacctgcatgctgggcacgtacacacaagacctcgacaagtttcacaccttcccccaaacttcaattggggttgaagcacttggcgaagaaaagggatgtggccaaggacttggagacaaaccaccaatcccattttggcaactaagctccttctctcctttctagtttccttcttgctttcttcctataacttgaggcatgtagttcctctctggttgctctccaggctattactggttgctttcctgaggcaaagaatggtatctgaaatccccagtgggtgaggagaaagtcccacaggctaaaagagaatcacccaggaagatggcagagagcaagggcacactcaggaagatggcagagagcaagggcagtcatctggcttcctagtagagcttctagtcttgcttctggaagtgttggttgtttgggaaataaaactattttctaaaaaaaaaaaaaaaaaaaa

SEQ ID NO: 2 - protein, 175 aa, mouse
QDQESPLRKHCLAPSGSPQAELQGTAHQEGIMGFLKFSPFLVVSILLLYQACSLQAVPLRSILGSSPGMATLSEEVRLLAAALVQDYMGMKPRELEQEEEQEAEGSSLDSPRSKRCGNLTTCMLGTYTQDLDLFHTFPQTSIGVEALGGEKGCGQGLGDLPPIPFWQLSSSFSPF

SEQ ID NO: 3 - protein, 23 aa, Mouse
CGNLTTCMLGTYTQDLDKFHTFP

SEQ ID NO: 4 - protein, 31 aa, Mouse
GGNLTTCMLGTYQDLDKFHTFPQTSIGVEAL

SEQ ID NO: 5 - DNA, 18 bp, Mouse
agagtcaccgcttcgcaa

SEQ ID NO: 6 - DNA, 22 bp, mouse
ccagagaggaactacatgcatc

SEQ ID NO: 7 - DNA, 18 bp, salmon
gcaagcaagatccacatg

SEQ ID NO: 8 - DNA, 22 bp, salmon
agagcaaccgctatgcaagcta

SEQ ID NO: 9 - DNA, 39 bp, mouse
caaggatcaagagtcaccgcttcgcaagcactgcctggc

(19) United States
(12) Patent Application Publication
(10) Pub. No.: US 2003/0114383 A1
(43) Pub. Date: Jun. 19, 2003

(54) Calcitonin-like sequence expressed by gonadotropes of the anterior pituitary

(76) Inventor: Girish Shah, Amarillo, TX (US)

Correspondence Address: JONES, TULLAR & COOPER, P.C., P.O. BOX 2266 EADS STATION, ARLINGTON, VA, 22202

(21) Appl. No.: 10/285,688

(22) Filed: Nov. 1, 2002

Related U.S. Application Data
(60) Provisional application No. 60/330,838, filed on Nov. 1, 2001.
(60) Provisional application No. 60/331,398, filed on Nov. 15, 2001.

(51) Int. Cl. C07K 14/575; A61K 38/24; C12P 21/02; C12N 5/06; C07H 21/04

(52) U.S. Cl. 514/12; 530/399; 536/23.5; 435/69.4; 435/320.1; 435/325

(57) ABSTRACT

A cDNA sequence has been identified for an anterior pituitary-derived peptide (pit-CT) produced and secreted by the pituitary cells. The pit-CT has substantial sequence homology to calcitonin (CT) and has biological properties similar to salmon calcitonin (SCT). The pit-CT may be used to treat various diseases, such as osteoporosis, Pagets, and Prolactinoma.

### CROSS-REFERENCE TO RELATED APPLICATIONS

This application claims the benefit, under 35 U.S.C. 119(e), of U.S. Provisional Application No. 60/330,838 filed Nov. 1, 2001 and U.S. Provisional Application No. 60/331,398 filed Nov. 15, 2001, the contents of which are incorporated herein by reference.

### BACKGROUND OF THE INVENTION

1. Field of the Invention

The present invention relates generally to the treatment of various diseases, such as osteoporosis, Pagets, and prolactinoma, using an anterior pituitary-derived peptide (herein referred to as “pit-CT”) which has substantial sequence homology to calcitonin (herein referred to as “CT”) and has biological properties similar to a calcitonin-like peptide derived from salmon, commonly known as salmon calcitonin (herein referred to as “SCT”).

2. Description of Related Art

Osteoporosis is a major health problem that affects more than 25 million women in the United States alone and potentially 200 million worldwide. The disease is characterized by diminished structural integrity of the skeleton, which results in an increased risk of fracture. Osteoporosis is a condition that develops silently over a period of years, eventually progressing to a point where a fracture can easily occur causing pain and disability. The post-menopausal female population has the highest incidence of osteoporosis and the highest rate of morbidity and mortality due to this disease. The National Osteoporosis Foundation in the U.S. estimates that osteoporosis is responsible for approximately 1.5 million fractures in the U.S. alone. Hip fractures lead to the most serious consequences with one in five hip fracture patients dying of complications within a year after the fracture and one in four requiring long-term care. The National Osteoporosis Foundation in the U.S. estimates that the cost of treating osteoporosis is approximately $14 billion annually in the U.S. alone.

Current drugs available for the treatment of post-menopausal osteoporosis include salmon calcitonin (SCT), a calcium-regulating peptide from salmon. Calcitonins (CT) are a group of polypeptide hormones containing 32 amino acid residues which all share an amino terminal ring structure with cysteines at 1 and 7 linked by a disulfide bridge, as well as carboxyterminal prolinamide. The CT-like peptide from salmon (SCT) has a potent effect on bone remodeling and consequently has been widely used as a therapy for the treatment of osteoporosis. Although SCT is effective in the short term, it is not efficacious for long term treatment because it is a peptide from fish. Long-term treatment results in the generation of antibodies against the peptide, resulting in the drug becoming ineffective. Consequently, one-term treatment with salmon calcitonin (SCT) is ineffective.

Human parathyroid calcitonin was widely used as a treatment for osteoporosis in the past, but its effect on the disease was marginal and only transient.

Current routes of administration of salmon calcitonin (SCT) are through intramuscular injection and intranasal administration as an aerosol. Intramuscular injection is not accepted by a large number of patients due to the pain involved and the assistance needed in injection.

Estrogen therapy is also currently available for the treatment of post-menopausal osteoporosis. Although more effective than salmon calcitonin (SCT), it is associated with increased risk of breast cancer thus preventing the widespread acceptance of this form of therapy. Additionally, estrogen therapy has very little effect on rebuilding bone, but retards bone loss in many patients.

New therapeutic drugs called amino bisphosphonates are gaining acceptance. The drug is deposited in the bone structure, thereby reinforcing the bones. The drug is expected to perform better than currently available therapeutics and has been shown to be effective in decreasing fractures in slightly less than half the patients treated. However, since the method of action of the drug is not through natural bone remodeling, but through deposition of the drug in the bone structure, it is unclear whether there will be any side effects associated with its long-term use. Other drugs in development include derivatives of estrogen, which has decreased potential to induce breast cancer, human parathyroid hormone and nasal formulation of salmon calcitonin. However, currently available clinical data indicate that these drug candidates are effective only in a smaller, although sizable, population of the patients, leaving a critical need to develop newer drugs that are more effective in the treatment of this disease that affects a large section of older women.

Pagets disease of the bone is a chronic disorder that typically results in enlarged and deformed bones. The cause of the disease is unknown. Excessive bone breakdown and formation cause the bone to be dense, but fragile. As a result, bone pain, arthritis, noticeable deformities and fracture can occur. Pagets disease manifests in up to 3% of the population in the U.S., but is rarely diagnosed in people less than 40 years of age. Bone pain is the most common symptom and the pain may occur in any bone affected by the disease and often localizes to areas adjacent to the joints. Headache and hearing loss may occur when the disease affects the skull or a curvature of the spine may occur in advance cases. The only approved therapies for Pagets disease are salmon calcitonin (SCT), both injectable and nasal formulations, and bis-phosphonates.

Salmon calcitonin (SCT) also has distinct analgesic properties in patients suffering from osteoporosis and Pagets disease.

Prolactinoma is a tumor of the pituitary, a neuroendocrine gland situated at the base of the brain. The tumor characteristically secretes excessive amounts of the hormone prolactin, which may have multiple functions, the most predominant one being involved in the production of milk in females. The tumor varies in size from microscopic to as large as several centimeters in diameter. Prolactinomas occur most commonly in those under 40 years of age. The incidence is 3 out of 10,000 in males and 1 out of 1000 in females. The disease causes infertility, a milky discharge from breasts, absence of menstrual periods and osteoporosis in women. In men, it can cause impotence, loss of libido and infertility. The tumor rarely needs surgical removal and the disease is commonly treated with dopamine agonists like bromocriptine. However, a high incidence of side effects, a short duration of action, and a lack of effectiveness in some patients limit its use. Cabergoline, another dopamine agonist, seems to be better tolerated than bromocriptine and is undergoing clinical trials.

CT-like immunoreactivity (CTI) is widely distributed in the central nervous system (CNS) and the pituitary gland of various mammalian species including rats and humans. Receptors recognizing salmon calcitonin (SCT) have been detected in specific regions of rat brain and the anterior pituitary (AP) gland, and complementary cDNAs for two such receptors have been cloned from a rat brain cDNA library. Supporting the physiological relevance of CT actions in the AP gland are the findings of the presence of salmon calcitonin-like immuno-reactivity (SCTI) and human calcitonin-like immunoreactivity (HCTI) in rat and human hypothalami and the pituitary glands by several investigators. The evidence for the synthesis and secretion of CT-like immunoreactive peptides by primary cultures of the rat AP gland has also been presented. Pituitary derived calcitonin may share antigenic sites with human, or rat, calcitonin and SCT since antisera raised against these peptides immunoprecipitate molecules of similar electrophoretic mobility from AP cell lysates. Using GCT1, an anti-SCT serum generated by the present inventor and disclosed in Shah et al, Endocrinology, 125: 61-67, 1989, CTI is selectively localized in gonadotropes, and not in the thyrotropes, somatotropes, lactotropes, corticotropes or folliculo-stellate cells of rat AP gland.

Previous finding have shown that exogenously added salmon calcitonin (SCT) significantly attenuates prolactin (PRL) release from perifused rat AP cells without altering the secretion of growth hormone (GH), follicle-stimulated hormone (FSH), luteinizing hormone (LH) or thyroid stimulating hormone (TSH). Salmon calcitonin (SCT) is also a potent inhibitor of PRL gene transcription and lactotrope cell proliferation in rats. Interestingly, the addition of GCT1-anti-SCT serum immunoneutralizes endogenous calcitonin stimulates PRL release from cultured rat pituitary cells and raises serum PRL levels in conscious ovariectomized rats. These results raise a possibility that gonadotrope-derived GCT1-immunoreactive CT is a paracrine inhibitor of lactotrope function. Since the molecular sequence of a gonadotrope-derived calcitonin-like peptide that regulates lactotrope function has not been determined, the identity of this important regulatory-peptide would be of great importance.

### BRIEF SUMMARY OF THE INVENTION

A cDNA sequence has been identified for an anterior pituitary-derived peptide (pit-CT) produced and secreted by the pituitary cells. The cDNA from the pit-CT is cloned from a mouse gonadotrope-derived LβT2 cell line and its sequence shows close homology to mouse calcitonin mRNA. The mRNA from the pit-CT has been localized to gonadotropes, and the encoded peptide regulates lactotrope function.

Analysis of the amino acid sequence of pit-CT has revealed that pit-CT shares many structural similarities with salmon calcitonin (SCT). Furthermore, many of the biological properties of the pit-CT resemble salmon calcitonin (SCT). This anterior pituitary-derived peptide (pit-CT) may be the mammalian equivalent of salmon calcitonin (SCT) or it is a closely-related peptide. Hence, pit-CT would have a strong effect on preventing bone demineralization in post-menopausal women and will be a novel therapeutic for the treatment of osteoporosis.

Unlike salmon calcitonin (SCT), which loses its activity upon long-term administration due to the body making antibodies against the peptide, the new pit-CT will not generate an antibody response thereby imparting long-term activity against osteoporosis.

Salmon calcitonin (SCT) also has distinct analgesic properties in patients suffering from osteoporosis and Pagets disease. Hence, pit-CT is expected to have the same analgesic properties in these patients and would be a distinct advantage over non-calcitonin based therapies.

Pit-CT is a very potent inhibitor of prolactin secretion by the pituitary of rats. This property makes the pit-CT a potent therapeutic candidate for the treatment of pituitary Prolactinoma. It is likely that unlike neuroactive dopamine agonists, pit-CT, which is a natural mammalian peptide, including a human peptide, will be better tolerated by patients suffering from Prolactinoma and associated conditions, especially infertility in younger mates.

Pit-CT could also have utility in the treatment of certain female infertility conditions arising out of high prolactin levels in their blood. Multiple applications of the same drug for the treatment of different disease conditions is a highly desirable property in any drug, since it significantly reduces the cost of development of the drug.

### BRIEF DESCRIPTION OF THE SEVERAL VIEWS OF THE DRAWINGS

The features and advantages of the present invention will become apparent from the following detailed description of a preferred embodiment thereof, taken in conjunction with the accompanying drawings, in which:

FIG. 1 is the nucleotide sequence listing (SEQ. ID No. 1) for an isolated DNA molecule encoding a peptide produced and secreted by the anterior pituitary cells (pit-CT);

FIG. 2 is the amino acid sequence listing (SEQ. ID No. 2) for an isolated DNA molecule encoding a peptide produced and secreted by the anterior pituitary cells (pit-CT);

FIG. 3 is the amino acid sequence listing (SEQ. ID No. 3) for an isolated DNA molecule encoding a peptide produced and secreted by the anterior pituitary cells (pit-CT);

FIG. 4 is the amino acid sequence listing (SEQ. ID No. 4) for an isolated DNA molecule encoding a peptide produced and secreted by the anterior pituitary cells (pit-CT);

FIG. 5 shows a comparison of sequence of pit-CT mRNA (pit-CT/c) and mouse CT mRNA (MMCALCIT);

FIG. 6 is a graph showing the overexpression of CT mRNA in stable CT.U6 transfectants;

FIG. 7 shows a typical profile of Western blotting of CT.U6 and parental LβT2 cell extracts;

FIG. 8 shows CTI-ICC of LβT2 cells and CT.U6 transfectants (A and B);

FIG. 9 is a graph showing that a co-culture with CT.U6 cells dramatically attenuates PRL release from GGH3 cells;

FIG. 10 is a graph showing that a co-culture with CT.U6 cells causes a marked decrease in PRL mRNA abundance of GGH3 cells;

FIG. 11 is a graph showing that co-culture with CT.U6 cells causes a dramatic decline in DNA synthesis of GH3 cells;

FIG. 12 shows a co-localization of CT mRNA with β-LH mRNA in rat AP gland; and

FIG. 13 shows a localization of CT and PRL mRNAs in rat AP gland.

### DETAILED DESCRIPTION OF THE INVENTION

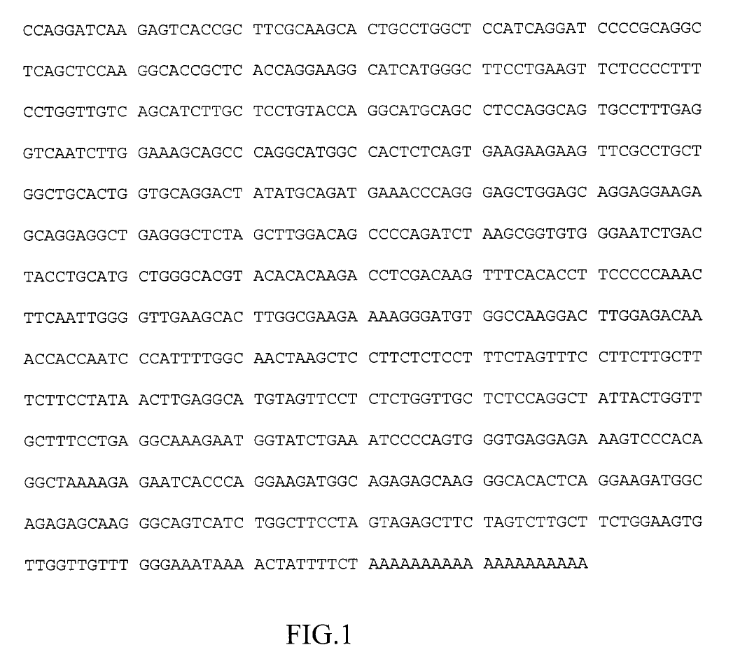

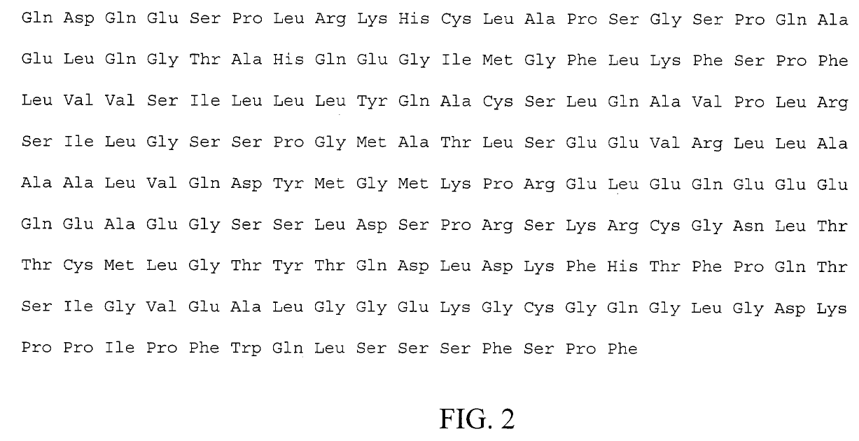

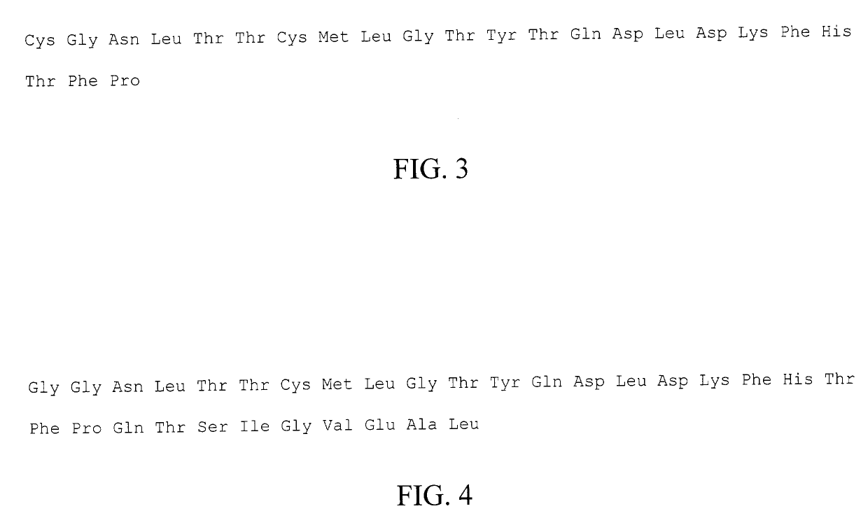

An anterior pituitary-derived peptide (herein referred to as “pit-CT”) and its human homologue peptides are made using standard recombinant DNA technologies. The sequence encoding pit-CT amino acid sequence is cloned into a mammalian expression vector, for example, pRC/CMV2 from Invitrogen, Carlsbard, Calif., USA. One could use a variety of commercially available vectors or proprietary vectors for expressing pit-CT. Also Chinese hamster ovary (CHO) cells and similar cell lines can be used for expressing the peptide. Alternate forms of expressing pit-CT include expression in bacteria using bacterial vectors, in yeast or in insect cells using baculovirus vectors or similar vectors. In fact, a variety of published methods used for expressing proteins can be used for the purpose. The cDNA sequence shown in FIG. 1 and SEQ. ID No. 1 is cloned from a mouse anterior pituitary cell line. The amino acid sequence shown in FIG. 2 and SEQ. ID No. 2 is derived from SEQ. ID. No. 1 by translation. The amino acid sequences in FIG. 3, which corresponds to SEQ. ID No. 3, and FIG. 4, which corresponds to SEQ. ID. No 4, are sequences of the mature peptide that has biological effects as described herein. SEQ. ID No. 3 and SEQ. ID No. 4 are derived from SEQ. ID No. 2. The nucleotides of SEQ. ID No. 1 or the amino acids of SEQ. ID No. 2, SEQ. ID No. 3, or SEQ. ID No. 4 are cloned into a suitable vector. The transcription of the DNA sequence is preferably under the control of the cytomegalo virus (CMV) promoter of the vector. The vector is preferably transfected into COS7 cells, a kidney cell line, using Lipofectin from Life Technologies, Maryland, USA and the transfected cells are preferably selected using hygromycin as described by the manufacturer. Single clone of cells producing high levels of pit-CT are selected. The level of expression of the pit-CT by the various clones is determined by Western blot as described hereafter.

Clones producing high levels of pit-CT are scaled up and the cells preferably harvested by centrifugation and lysed by gentle agitation in the presence of 0.5% NP 40 detergent. One could use a variety of standard methods to disrupt the cells and release pit-CT. The lysed cells are clarified and the supernatant is preferably passed through an immunoaffinity column of anti-pit-CT monoclonal antibodies coupled to Sephacryl 6B. One could use a variety of matrices. The clarified lysate is preferably passed slowly through the immunoaffinity column, washed with phosphate buffered saline at a pH of 7.4 and eluted with pH 4.0 buffer and quickly neutralized using alkaline phosphate buffer to neutral pH. The eluted pit-CT is preferably dialyzed against water or saline and lyophilized prior to use. One can also use other standard methods for purifying the pit-CT including gel filtration, ion-exchange chromatography, hydrophobic chromatography and other published methods.

A second preferred method of producing pit-CT is by automated synthesis using a peptide synthesizer from Applied Biosystems, California, USA. The peptide is preferably cleaved from the support and deprotected as prescribed by the manufacturer. The peptide is preferably purified on a C-3 or C-18 reverse phase column using standard high-pressure liquid chromatography. The peptide is preferably dialyzed or desalted against water or saline and lyophilized prior to use.

Conjugating polyethylene glycol (PEG) to the pit-CT through the carboxyl terminal can make a particular PEG-conjugated formulation that will allow the pit-CT to have longer half-life in blood of patients. Standard published methods for conjugating PEG to peptides are used for this purpose.

One modality of treating osteoporosis and Pagets disease is by administration of salmon calcitonin (SCT) either as an intramuscular injection or as formulations that can be administered nasally as an aerosol. The pit-CT or its human homologue or its PEG-conjugated formulation is reconstituted in saline and injected intramuscularly or administered as an aerosol formulation nasally. The doses have to be calibrated based on the response of the drug to the disease. Generally pit-CT has to be administered.

Pit-CT can be used for the treatment of Prolactinoma, a common tumor of the pituitary. Reconstituted pit-CT or its PEG-conjugated formulation or pit-CT as an aerosol formulation is administered to the patient as a treatment for Prolactinoma. The doses have to be determined for the patients depending on the response.

### EXAMPLE

Pit-CT cDNA from a mouse gonadotrope LβT2 cell line has been cloned using reverse transcription polymerase chain reaction (RT-PCR) and rapid amplification of cDNA ends (RACE) techniques. Alignment of nucleotide sequences of pit-CT and mouse CT reveals greater than 99% homology between the sequences. The pit-CT cDNA was ligated into a mammalian expression vector, and the construct was transfected into LβT2 cells. Two stable transfectant cell lines, CT.U6/A and B, were obtained by selection in G418, an antibiotic. Subsequent S1-nucleas protection assay and immunocyto-chemistry results have shown that: (1) pit-CT expressed by CT.U6 cell lines immunoreacted with GCT1-anti-SCT serum; (2) secretion of CT.U6 cells inhibited prolactin (PRL) release, PRL mRNA abundance and DNA synthesis of PRL-screening GGH3 cells (a rat pituitary cell line); and (3) CT.U6-induced inhibition was abolished by GCT1-anti-SCT serum. A riboprobe was generated from the cloned pit-CT cDNA and localized CT mRNA expression in gonadotropes of rat AP gland by in situ hybridization histochemistry.

Therefore, it has been determined that pit-CT mRNA is closely homologous to mouse CT mRNA; it is expressed by gonadotropes of the rat AP gland; and the peptide may significantly affect lactotrope function by inhibiting PRL release and cell proliferation.

Cell Lines

LβT2, a gonadotrope-derived mouse cell line that secretes α as well as β subunits of luteinizing hormone (LH) were maintained in a growth medium prepared in Dulbecco's modified eagle's medium (DMEM) containing 4.5 mg/ml glucose and supplemented with 10% fetal calf serum.

Preparation of RNA and RT-PCR

Total RNA from LβT2 cells was extracted using a RNeasy Mini Kit from QIAGEN, Valencia, Calif., USA. One microgram total RNA was used for reverse transcription (RT). Oligo dT primer annealing and reverse transcription were performed using Superscript II reverse transcriptase according to the manufacture's protocol (GIBCO-BRL, Gaithersburg, Md., USA).

Polymerase Chain Reaction (PCR)

Reverse transcribed cDNA was amplified with a polymerase chain reaction (PCR) reagent kit purchased from GIBCO-BRL. Since human calcitonin-like (HCT-like), as well as SCT-like, peptides have been detected in the AP gland, two sets of primer pairs were used derived from either mouse (mCT) or SCT cDNA sequences: mCT-forward: 5′-agagtcaccgcttcgcaa-3′ (SEQ. ID. No. 5); mCT-reverse: 5′-ccagagaggaactacatgcatc-3′ (SEQ. ID. No. 6); SCT-forward: 5′-gcaagcaagatccacatg-3′ (SEQ. ID. No. 7); SCT-reverse: 5′agagcaaccgctatgcaagcta-3′ (SEQ. ID. No. 8).

The hot start method was employed to minimize non-specific amplification. The amplified product(s) was fractionated on a 1% agarose gel, the bands were cut, and the DNA was extracted and subcloned in pGen-T vector (Promega Laboratories, Milwaukee, Wis., USA). The recombinant plamids were sent for DNA sequencing.

RACE and Screening

The first series of PCR reaction yielded two clones that were highly homologous to the mouse CT sequence. Since these sequences were partial, RACE reaction was employed to obtain longer sequences. A gene specific primer (GSP) 5′-caaggatcaagagtcaccgcttcgcaagcactg cctggc-3′ (SEQ. ID. No. 9), which was synthetically-derived from a previously cloned partial mouse pit-CT cDNA sequence, and a universal primer mix (CLONTECH), which is a mixture of synthetic oligonucleotides available from Clonetech Inc., were used for 3′-RACE. The reaction was first cycled 5 times (5 s, 94° C.; 3 min. 72° C.), and cycled 5 times again (5 s, 94° C.; 10 s, 70° C.; 3 min, 72° C.) based on the manufacturer's recommendations, and then subjected to 30 cycles of PCR (5 s, 94° C.; 10 s, 60° C.; 2 min, 72° C.). PCR products were purified with a DNA purification kit (Bio-RAD, Hercules, Calif., USA) and ligated into the pGEM-T vector. Plasmid DNA from several clones was prepared and identified by Southern blotting using the partial CT cDNA probe. Positive RACE clones were sent for DNA sequencing.

Expression of Recombinant pit-CT in LβT2 Cells

Pit-CT cDNA insert was cloned downstream of the cyromegalovirus promoter in pcDNA3.1/Myc-His(+)B vector (Invitrogen, San Diego, Calif., USA). The presence and orientation of the insert in the recombinant plasmid (pcDNA3.1-CT) was verified by digestion with appropriate restriction enzymes as well as by DNA sequencing of the insert. Vector pcDNA3.1 has two distinct C-terminal epitopes to detect the recombinant proteins. The C-terminal of the expressed protein will carry a c-myc epitope that can be identified by anti-myc antibody. This epitope is followed by a polyhistidine tag, which can be identified by anti-His (C-term) antibody.

The recombinant plasmid was then used for transfection in LβT2 cells. LβT2 cells in mid-to-late log phase were harvested and resuspended in ice-cold PBS at 10 million cells/ml, and mixed with plasmid DNA (recombinant plasmid pcDNA 3.1-CT or vehicle plasmid pcDNA 3.1, 30 μg/ml) in a electroporation cuvette, and electroporated at 800 μF and 250V (Gene Pulser II, Bio-RAD). Transfected cells were incubated in a 6-well plate with DMEM for 48 hours and then selected with 400 μg/ml G418. Individual colonies were selected after four weeks of culture, dispersed with trypsin/EDTA and propagated further into flesh flasks. Two cell colonies, CT.U6/A and CT.U6/B, displaying the highest CTI secretion were further investigated.

Detection of CT mRNA in CT-Transfectants

The cell lines LβT2 (parental), V (carrying vehicle plasmid), CT.U6/A and CT.U6/B were cultured as described above. Three hundred thousand cells of each of these cell lines were seeded individually into 100 mm dishes, and were grown to exponential phase. The total RNA from these cell lines was extracted as previously described, and was used to determine CT mRNA abundance by S1-nuclease protection assay.

S1-Nuclease Protection Assay

Uridine triphosphate (UTP)-labeled antisense riboprobes of pit-CT, PRL and β-actin were generated using T7 RNA polymerase (Promega) and appropriate linearized DNA templates. Total RNA (20 μg) was incubated with the appropriate riboprobe for 18 hours at 45 ° C. Following this, the samples were treated with 51-nuclease for 30 min at 37 ° C. The protected RNA was precipitated and fractioned on 4.5% polyacrylamide gel with 8 M urea. The gel was then dried and autoradio-graphed. Each experiment was repeated three separate times.

Secretion of CT1in the Conditioned Media

Levels of CTI and PRL in the conditioned media of LβT2 cells and CT-transfectants were determined by radioimmunoassay (RIA). The PRL RIA used the reagents provided by the National Institute of Diabetes, Digestive and Kidney diseases (NIDDK), and the assay protocol recommended by the NIDDK was followed. CTI was determined as described previously. All samples from this series of experiments were concurrently run in duplicate in the same RIA. Protein A (IgG Sorb) was used for the separation of bound from free hormone antigen. The experiments were repeated at least three separate times, and data from all the experiments were pooled for statistical analysis.

CT Immunocytochemistry (ICC)

Approximately 10,000 LβT2 cells of CT-transfectants were plated onto polylysine-coated microscope slides. The cells were incubated for 18 hours at 37 ° C., washed with phosphate-buffered saline (PBS) and fixed for 1 hour in Zamboni's solution. The fixed cells were processed for pit-CT ICC as previously described except that the second antibody was conjugated to horse-radish peroxidase (HRP) and therefore the color was developed using diaminobenzidine tetrahydrochloride (DAB) as a substrate. The negative controls were treated with primary antiserum that was preincubated with 1 μM SCT at 37 ° C. for 1 hour. The experiments were repeated two more times.

Crude cell lysates from the parental LβT2 cells and CT.U6 (A and B) were prepared as previously described. In brief, 10 million cells from each cell line were homogenized in Buffer A {25 mM Tris, pH 7.4 containing 10% glycerol, 1% Nonidet P-40, 50 mM NaF and freshly supplemented with 10 mM sodium pyrophosphate (PPi), 1 mM sodium vanadate, leupeptin (10 μg/ml), aprotinin (5 μg/ml), and phenylmethylsulfonyl fluoride (1 mM)}. Nuclear fraction and debris were separated by centrifugation at 2000×g for 10 min at 4 ° C., and the supernatant was used for Western blot analysis. Protein concentrations of cell lysates were determined by the method of Bradford (Bio-RAD). The lysates were then boiled for 5 min in 2× Lemmli solution containing 20 mM dithiothreitol (DTT), and 50 μg protein per lane were loaded onto 12.5% SDS-polyacrylamide gel. The separated proteins were electrically transferred to nitrocellulose, and the blots were incubated with previously characterized GCT1 rabbit anti-SCT serum (1:500) as well as mouse anti-histamine serum (C-terminal, 1:3000, Invitrogen, San Diego, Calif., USA) for 18 hours at 4 ° C. Following three washes, the membranes were incubates with either anti-rabbit or anti-mouse IgG-HRP (1:1000). Following three successive washes, the immune complexes were visualized using Western blot ECL detection system (Radiochemical Center, Amersham). The same experiment was repeated one more time.

Biological Activity of Recombinant Pit-CT Co-Culture of CT-Transfectants and GGH3 Cells and its Effect on PRL Secretion, PRL mRNA Abundance and DNA Synthesis of GGH3 Cells

To further examine the role of pituitary-derived CT in lactotrope function, a two-tier co-culture system was developed where b 1×10⁵ LβT2 cells or CT transfectants (CT.U6/A or CT.U6/B) are cultured separately in an upper chamber insert, whereas 2×10⁵ GGH3 cells per well (target cells) are cultured in a 12-well plate. During the experimental period, the upper chamber is inserted on top of a well of a 12-well plate containing GGH3 cells so that CT-secretors in the upper chamber do not come in direct contact with GGH3 cells in the lower chamber but are exposed to their secretions. The GGH3 cells in each set of the experiment were treated as follows: (1) vehicle control where the upper chamber contained GGH3 cells instead of CT-secretors+10 μl non-immune serum (NIS) in the lower chamber; (2) upper chamber contained either LβT2 or CT.U6 (A or B), and 10 μl NIS were added in the lower chamber; and (3) upper chamber contained either LβT2, CT.U6/A or CT.U6/B cells, and 10 μl anti-SCT serum were added to the medium in the lower chamber. After the incubation period of 24 hours, either the conditioned media or GGH3 cells in the lower chamber were harvested. The conditioned media were analyzed for PRL by RIA. The cell lysates were used to analyze PRL mRNA abundance as described in the S1-nuclase protection assay. Each data point was run in triplicate and the data from three independent but similar experiments were obtained.

The results on PRL release are expressed as ng PRL released by 100,000 cells over 24 hours. The results on PRL mRNA were digitized, normalized with β-actin mRNA and expressed as normalized densitometric units. The data from all experiments were pooled and expressed as means ±S.E.M. The results were statistically evaluated by one-way ANOVA and the significance was derived by Newman-Keul's test.

[³H] Thymidine Incorporation of GGH3 Cells

GGH3 cells in log phase were seeded at 1×10⁵ cells/well in 1 ml complete medium in 12-well culture plates. The growth rate of cells was slowed down by overnight incubation in low-serum-containing medium, such as 2% fetal calf serum (FCS), followed by 2-hour incubation in serum-free basal medium. The cells were then co-incubated with CT-transfectants as described above for 24 hours. Four hours prior to the termination of the assay, the GGH3 cells in the lower chamber received [³H] thymidine (0.5 μCi/well). At the end of the incubation, the cells were washed twice with PBS containing 100 μM unlabeled thymidine, and solubilized in Triton X-100 (0.1% vol/vol in distilled water). The incorporated [3 H] thymidine was quantified by liquid scintillation counting. Each data point was run in quadruplicate and the date from three independent but similar experiments was obtained.

The results are express as d.p.m of [³H] thymidine incorporated per 1×10⁵ cells ±S.E.M. The data from all experiments were pooled and analyzed by one-way ANOVA and the level of significance was derived from Newman-Keul's test.

Localization of Pit-CT mRNA in Rat AP Gland: in situ Hybridization Histochemistry (ISH)

Sense/anti-sense digoxigenin-labeled pit-CT riboprobes are prepared in the following manner. Plasmid containing partial CT.U6 (86-580) was linearized, and antisense riboprobes was transcribed using T7 RNA polymerase. Similarly, a sense riboprobe was generated using SP6 RNA polymerase. Digoxigenin 11-UTP (Boehringer Mannheim, Indianapolis, Ind., USA) was used in both transcription reactions, and the manufacturer's instructions were followed. The reaction mixtures were digested with RNAse-free DNAse (Bohringer), the riboprobes were extracted with phenol/chloroform, and purified on TE microselect-D G-50 spin columns (5 Prime-3 Prime, Inc., Boulder, Colo., USA).

LH-β and PRL cDNA-rhodamine probes are prepared in the following manner. cDNA inserts for rat LH-β or rat PR1 were labeled with tetramethyl rhodamine-6-dUTP by random primer labeling using klenow fragment of DNA polymerase, and the probes were purified on TE Microselect-D G-50 spin columns.

Next, double in situ hybridization histochemistry (ISH) is preformed in the following manner. The rat AP glands were rapidly frozen by submersion in an isopentane-dry CO2 bath after mounting in the embedding medium (OCT compound, Tissu-Tek, Miles Laboratories, Elkhart, Ind., USA). The frozen tissues were sliced to 5-10 μm thick sections and thaw-mounted on Superfrost plus glass slides (Fisher Scientific, Pittsburgh, Pa., USA). The sections were stored frozen at −70 ° C. until ISH analysis.

The frozen tissue sections were rapidly thawed, washed with PBS at 4° C., and fixed in 4% papformaldehyde-PBS (pH 7.2) for 10 min. The double ISH procedure was performed using antisense pit-CT RNA and LH-β or PRL cDNA probes as described before. Serial sections of the specimens were concurrently probed with sense probes, which served as negative controls. The hybridization signal of CT mRNA was detected by incubating the hybridized sections with mouse anti-digoxigenin-FITC for 6 hours at 4 ° C., whereas the cDNA probes for LH-β or PRL contained fluorescent ribonucleotide and did not need additional processing. Three animals per group were used for these experiments. Sections from all animals were processed simultaneously. Two researchers independently evaluated the slides, scoring all slides at the same time to avoid comparing preparations that had been stored or exposed to UV-light for different periods of time. The sections, at least twelve/experiment from three different animals, were observed under a Nikon Optiphot microscope with epifluorescence attachment. The digital images were captured on a G3 Power PC computer by a Spot camera attached to the microscope and examined for co-localization between pit-CT mRNA and LH-β or PRL mRNA.

### RESULTS

Amplification and Sequencing of Pit-CT mRNA from LβT2 Cells

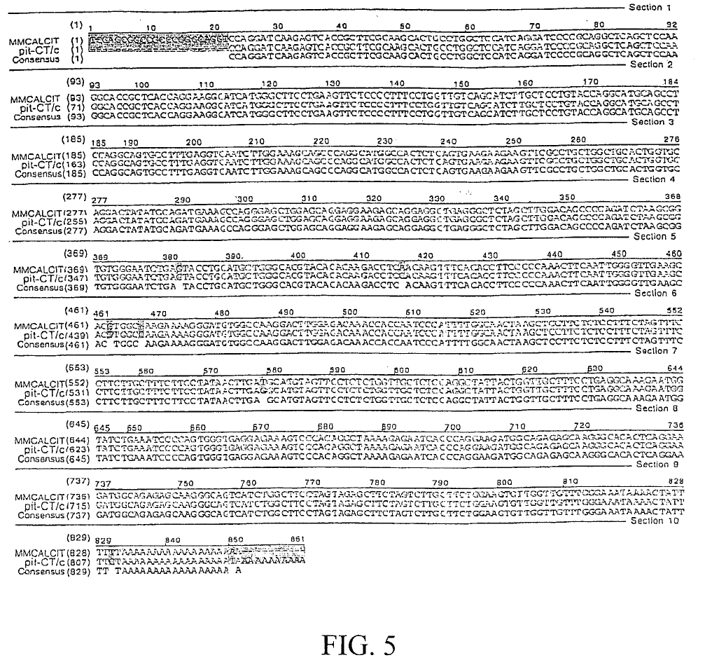

Initial PCR experiments with mCT amplimers yielded a partial cDNA of 564 bp length. The cDNA sequence displayed greater than 90% homology with mouse CT mRNA sequence (emb/X97991.1). Thereafter, the deduced amino acid sequence of the known mouse CT was aligned with partial pit-CT sequence, and the conserved region was used to design a specific primer for 3′-RACE. First-strand cDNA produced by RT with 3′-CDS primer and universal primer mix was used as a template for 3′-RACE. 3 ′-RACE products were ligated into pGEM-T vector. Nineteen clones were identified by Southern blotting and DNA sequencing. The alignment of nucleotide sequences revealed that a clone, pit-CT.U6, had greater than 99% homology with the 23-829 bp segment of mouse CT mRNA (emb/X97991.1), as shown in FIG. 5. Only seven out of 806 bases in the pit-CT mRNA sequence differed from mouse CT mRNA sequence. The mismatches were at positions 383, 418, 463, 469, 580, 831 and 851 of mouse CT sequence. Homology with rat CGRP sequence was much less and covered only 58-232 bp segment of rat α CGRP mRNA (emp/VO1229.1). Moreover, the presence of exon 4 in the pit-CT sequence, which is specific for CT but not CGRP, suggests that the CT gene of LβT2 cells transcribes CT, and not CGRP, mRNA.

Stable LβT2-CT Transfectants Express Pit-CT mRNA and GCT1-Immunoreactive CT

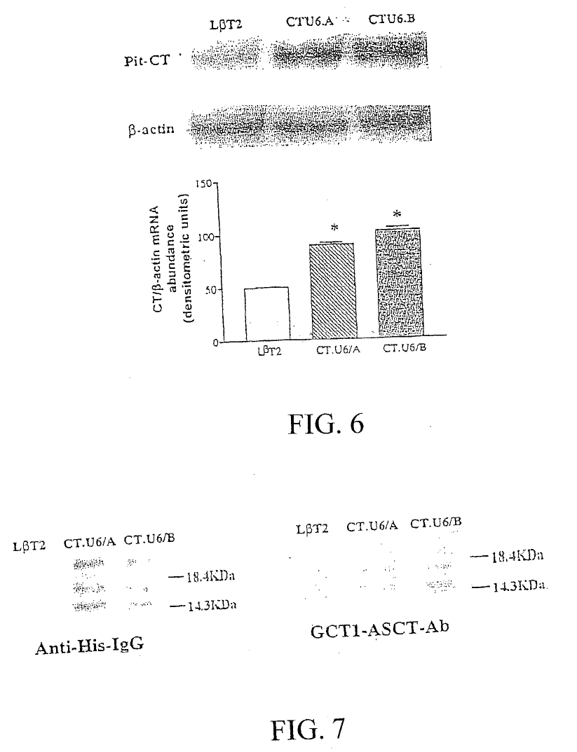

Cell clones CT.U6/A and CT.U6/B were obtained by selecting LβT2 transfectants with G418. The results from S1 -nuclease protection assay showed that CT.U6 (A and B) cells displayed markedly greater abundance of pit-CT mRNA than the parental LβT2 cells, as shown in FIG. 6. Relative densitometric value of pit-CT mRNA in CT.U6 cells increase by 97% over parental LβT2 cells. The data from three independent experiments were digitized to obtain relative densitometric units. Pooled data from these experiments showed an almost twofold increase in CT mRNA abundance of the transfectants as compared with parental LβT2 cells. The results are expressed as means ±S.E.M. densitometric units (n=6). The data were analyzed by one-way ANOVA and significance was derived by Newman-Keul's test.

Since p1DNA3.1/Myc-His(+)B vector expresses fusion protein, the recombinant pit-CT.U6 protein should also express fusion protein poly Myc-His. The results from Western blot analysis of transfectants reveal that anti-Hid antibody identified three major immunoreactive bands in CT.U6 lysates, as shown in FIG. 7. Fifty micrograms cell lysate proteins were size-fractioned on 12.5% SDS-polyacylamide gel. Following electric transfer, the nitrocellulose membranes were immunoblotted with anti-His antibody (Anti-His_IgG; left panel of FIG. 7) and blotting detection reagents. Positions of protein markers are indicated on the right panel of FIG. 7. Since pcDNA3.1/Myc-His(+)B vector expresses fusion protein, the expressed pit-CT.U6 peptide will be fused with poly Myc-His. Anti-His antibody detected two CT.U6 cell-specific immunoreactive bands. Interestingly, the same bands were also identified by GCT1-anti-SCT antibody. This suggests that fusion proteins in these two bands contain the encoded pit-CT peptide that cross-reacts with GCT1-anti-SCT serum. However, the band with the highest molecular size was also observed in control LβT2 cells which did not express the recombinant protein, and may be a plasmid-related band.

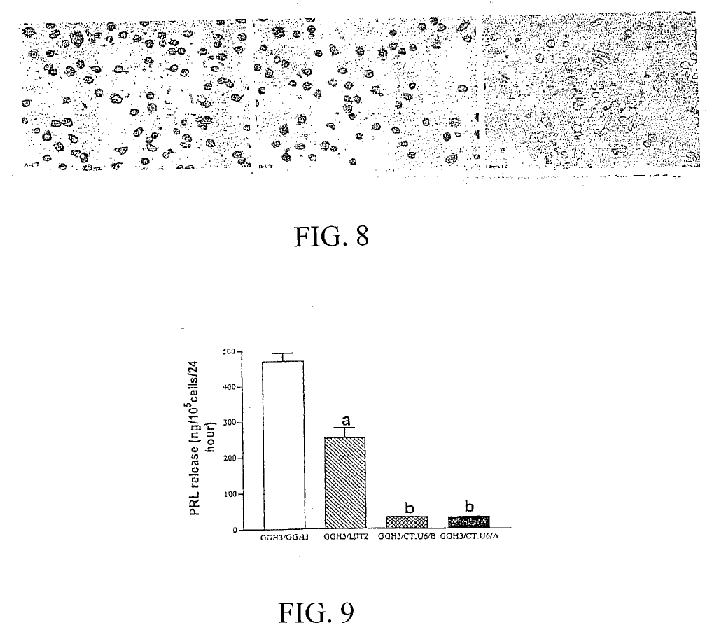

Consistent with the results from Western blot analysis ICC results also show that both CT.U6 clonal cell lines A and B stained strongly for CTI (GCT1). In contrast, control LβT2 cells were weakly positive, as seen in FIG. 8. LβT2 cells as well as CT.U6 A and B cell lines were processed for CT ICC as described above. GCT1-anti-SCT serum was used as primary antiserum. Both CT.U6 cell lines (A and B; left and middle panels of FIG. 8) stained intensely for CTI. In contrast, LβT2 cells (right panel of FIG. 8) stained only lightly under the same experimental conditions. Negative controls were preabsorbed antiserum did not display any staining. The experiment was repeated with three different cultures of LβT2 and CT.U6 (A and B).

Secretion of CTI by CT.U6 Cells

Results from CT RIA of spent media suggest that CT.U6 cell lines released 122.47 pg/100,000 cells of CT-Eq in 24 hours. The corresponding release from control LβT2 cells was very close to the detection limit of the assay (30 pg CT-Eq).

CT.U6 Cells Inhibit PRL mRNA and PRL Release from GCH3 Cells

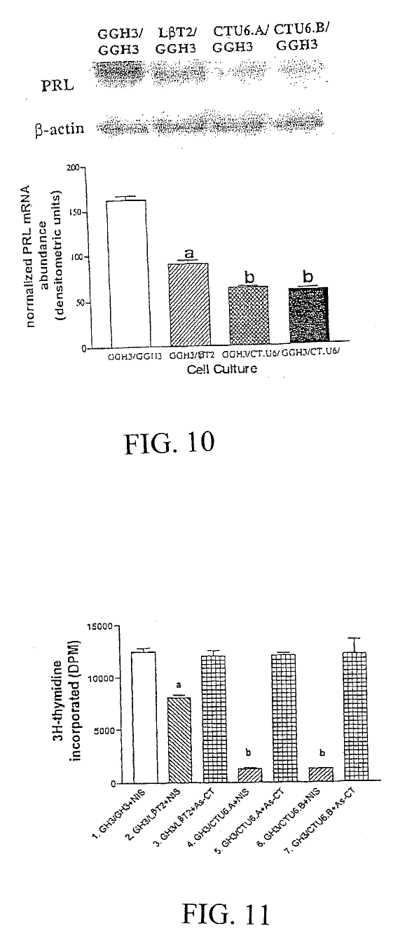

To test whether the actions of pit-CT on lactotrope function are consistent with previously demonstrated actions of GCT1-reactive CT, a two tier co-culture system was developed where GH3 cells received secretions of either LβT2 of CT.U6 cells but did not come in direct contact with them. As presented in FIG. 9 and FIG. 10, secretions of both CT.U6 cell lines A and B caused a dramatic inhibition in PRL secretion as well as PRL mRNA levels. Parental LβT2 cells also caused a decrease in PRL mRNA abundance and PRL release, but the decrease was smaller compared with that caused by CT.U6 cell lines. To obtain the data in FIG. 9, GGH3 and LβT2/CT.U6 cells were co-cultured with GGH3 cells in transwell culture plates as described above. Spent media were collected after 24 hours of co-cultures and analyzed for PRL by RIA. Controls were GGH3/GGH3 homologous co-cultures. Each data point was run in triplicate, and the experiment was repeated three times. The pooled results are presented as mean ng PRL released ±S.E.M. (n-9). The results were analyzed by one-way ANOVA and significance was derived by Newman-Keul's test. To obtain the data in FIG. 10, GGH3 and LβT2 or CT.U6 A and B cells were co-cultured with GGH3 cells in transwell culture plates as described above. The GGH3 cells were lysed, RNA was extracted and PRL mRNA abundance was determined by S1-nuclease protection assay. β-actin mRNA abundance was also measured.

Controls were GGH3/GGH3 homologous co-cultures. The results from three separate experiments were quantified by densitometry, normalized and are expressed as mean ±S.E.M. densitometric units (n--6). The data were statistically analyzed by one-way ANOVA, and significance was derived by Newman-Keul's test.

Secretions from CT.U6 Cells Inhibit DNA Synthesis of GC3 Cells: this Action is Reversed by GCT1-Anti-SCT Serum

Since exogenously added CT inhibited proliferation of lactotropes under in vitro as well as in vivo conditions, the effect of co-culture of CT-transfectants on DNA synthesis of GGH3 cells was tested. The results present in FIG. 11 show that [³H]thymidine incorporation in GGH3 cells co-cultured with CT.U6 cell lines (bars 4 and 6 in FIG. 11) was dramatically lower when compared with the vehicle controls (bar 1). In parallel experiments, control LβT2 cells were co-cultured with GGH3 cells. Similar to CT.U6 cells, LβT2 cells also decreased DNA synthesis of GGH3 cells (bar 2 in FIG. 11). However, this inhibition was much small as compared with that produced by CT-transfectants (bar 2 vs. bars 4 and 6 in FIG. 11). GCT1-anti-SCT antiserum almost abolished the inhibitory effect of LβT2 and CT.U5 cell lines on DNA synthesis of GGH3 cells (bars 3, 5, and 7 in FIG. 11). To obtain the data in FIG. 11, GGH3 and LβT2 or CT.U6 A and B cells were co-cultured with GGH3 cells in transwell culture plates as described above. In addition, the cells received either non-immune serum (NIS, 1:50) or GCT1-anti-SCT serum (As-CT, 1:50). The GGH3 cells were treated with 0.5 μCi [³H]thymidine during the last four hours. The cells were lysed and the incorporated [³H]thymidine was determined. Each experimental data point was run in quadruplicate, and the experiments were repeated three separate times. The results are expressed as mean [3H]thymidine incorporated ±S.E.M. (n=12). The results were further analyzed by one-way ANOVA and Newman-Keul's test.

Localization of Pit-CT mRNA in Gonadotropes of Rat AP Gland

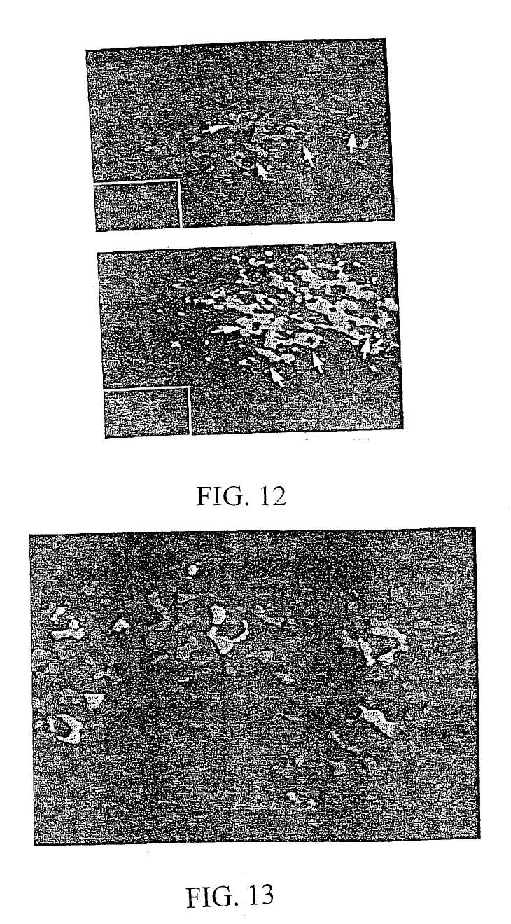

Since the pit-CT clone obtained from LβT2 cells expressed 91% homology with rat CT mRNA (gb/M26137.1), a partial pit-CT cDNA vector for riboprobe generation was constructed. The sequence of this pit-CT cDNA corresponded with the 4-493 bp segment of rat calcitonin mRNA (RCALC2), and displayed 92% homology. The digoxigenin-UTP-labeled anti-sense probe was used for in situ hybridization histochemistry with frozen sections of the AP glands obtained from cyclic female rats in the diestrous phase. Approximately 7-8% of total AP cells displayed pit-CT message, see the upper panel of FIG. 12. A similar distribution profile was also observed for LH-β mRNA, see the lower panel of FIG. 12, and co-localization of both these messages could be observed. Sense controls for CT as well as LH probes did not display significant staining, see the insets of FIG. 12, upper and lower panels. These results are consistent with previous findings using GCT1 and rat LH-β antisera. To obtain the data in FIG. 12, Fluorescent probes for β-LH mRNA (TRITC) and CT mRNA (FITC) were prepared as described above. Frozen rat AP sections (5 μm thick) were hybridized with these probes. Arrowheads in a typical micrograph depict co-localization of CT mRNA in the same cells. Sense controls are presented in the inset.

In another experiment, a relationship between CT cells and lactotropes was examined. The results presented in FIG. 13 show that CT mRNA-positive cells (green) were in apposition with PRL mRNA-positive cells (red). However, no co-localization of PRL and CT mRNA was observed. Again, sense controls did not display any staining. These findings are also in agreement with previously published results using GCT1-anti-SCT serum. To obtain the data in FIG. 13, the AP sections were hybridized simultaneously with PRL and CT mRNA probes as described above. A typical micrograph shows that the AP cells labels either for PRL mRNA or for CT mRNA, and co-localized of both mRNAs in the same cells was not observed. However, most CT mRNA cells seemed to have been surrounded by PRL mRNA cells.

### DISCUSSION

Although there have been reports of the expression of CT-like peptides in the pituitary gland, these results for the first time define the sequence of pituitary-derived CT. cDNA sequence of pit-CT showed greater than 99% homology with mouse CT mRNA sequence. It is conceivable that a few mismatches found in pit-CT sequence may have occurred due to spontaneous mutations known to occur in immortalized cell lines. It is also possible that Taq polymerase may have introduced mutations(s) during amplification. Additional studies with mouse AP glands will be necessary to explain these differences. However, the present results demonstrate that the mRNA sequence of pit-CT is closely homologous to mouse CT sequence, and the peptide encoded by the pit-CT mRNA immunoreacts with GCT1-ant-SCT serum. These results support the earlier observations that gonadotrope-derived CT immunoreacts with GCT1-anti-SCT serum.

Second, it was determined that pit-CT mRNA is localized in gonadotropes of rat AP gland. Using an anti-sense riboprobe derived from cloned pit-CT cDNA, the present results have shown that pit-CT mRNA is co-expressed with β-LH mRNA, and lactotropes (PRL mRNA-positive cells) display close anatomical apposition to pit-CT mRNA-positive cells. Both these results are consistent with earlier findings. Although the presence of SCT-like and HCT-like immunoreactive peptides in the AP gland has been reported by several investigators, there is only one report on the detection of rat CT mRNA in this organ. The present results are at variance with the earlier study that reported the lack of detectable CT mRNA in rat AP gland. It is conceivable that low copy numbers of pit-CT mRNA in rat AP gland and utilization of the less sensitive Northern blot technique may have been responsible for this discrepancy. Indeed, pit-CT mRNA in rat AP gland could not be detected by Northern blot analysis; however, the detection was possible with more advance techniques such as RT-PCR, S1-nuclease protection assay and in situ hybridization, which can detect single copy messages.

Third, it was determined that the translated product of pit-CT cDNA inhibits SCT, when administered either centrally or peripherally, inhibits PRL release in rats. In vitro experiments have extended these earlier findings by demonstrating that SCT acts directly at the level of lactotropes to inhibit PRL release, PRL gene transcription and lactotrope cell proliferation. A role for the endogenous peptide in these processes is demonstrated by the findings that immunoneutralization of pit-CT with GCT1 anti-SCT serum causes a significant increase in PRL release from cultured AP cells as well as in ovariectomized conscious rats. Consistent with these findings, the present results show that stable pit-CT transfectants secrete high concentrations of GCT1-reactive CTI, and they markedly inhibit PRL secretion, PRL mRNA abundance, and also attenuate proliferation of GGH3 cells. A role of pit-CT in these effects is implicated by the findings that GCT1-anti-SCT serum severely attenuates this inhibition.

There is accumulating evidence for the role of paracrine/autocrine peptides in the function of the AP gland. Different cell types display different proportions in the AP gland, and their relative proportions change continuously with changing hormonal environment. For example, there is a dramatic increase in lactotrope cell populations during pregnancy and lactation. There is also a drastic decrease in these populations with the cessation of lactation. Moreover, pituitary cell types respond to hormonal stimuli differently in different physiological conditions or hormonal milieu. The evidence suggest that signals provided by gonadal or neuroendocrine hormones to their target AP cells may be amplified or modulated by paracrine/ autocrine factors. For example, pituitary-derived vasoactive intestinal polypeptide (VIP) and galanin stimulate PRL secretion and also induce lactotrope proliferation. It has been suggested that several effects of estrogen on lactotrope function and proliferation are mediated by lactotrope-derived VIP and galanin. Similarly, studies suggest that CT inhibits PRL release and PRL gene transcription, and also attenuates thyrotropin-releasing hormone (TRH)- and suckling-induced PRL release and synthesis. CT is also a potent inhibitor of lactotrope proliferation. Expression of pit-CT is almost undetectable in early and mid-lactation but displays a dramatic increase in late lactation. Moreover, estrogen, which stimulate PRL synthesis and lactotrope proliferation, attenuate CT expression in the AP gland. Considering the secretion of CT by gonadotropes and the antagonistic functional relationships between gonatotropes and lactotropes, these results raise the strong possibility that gonadotropes may remodel the AP gland by modulating lactotrope cell number and function through the secretion of CT.

Although the present invention has been disclosed in terms of a preferred embodiment, it will be understood that numerous additional modifications and variations could be made thereto without departing from the scope of the invention as defined by the following claims:

What is claimed is:

1. An isolated nucleic acid sequence encoding a peptide produced and secreted by the pituitary cells, selected from the group consisting of:
a) a nucleic acid sequence having at least 90% homology with nucleotides of SEQ. ID. No. 1; and
b) a nucleic acid sequence encoding a peptide having an amino acid sequence which has at least 90% homology with the amino acids of SEQ. ID. No. 2 or SEQ. ID. No. 3 or SEQ. ID. No. 4.

2. An expression vector comprising the nucleic acid sequence of claim 1.

3. A host cell comprising the expression vector of claim 2.

4. The nucleic acid sequence of claim 1, wherein the nucleic acid sequence has at least 99% homology with nucleotides of SEQ. ID. No. 1.

5. The nucleic acid sequence of claim 4, wherein the nucleic acid sequence has nucleotides of SEQ. ID. No. 1.

6. The nucleic acid sequence of claim 1, which encodes a peptide having an amino acid sequence which has at least 90% homology with the amino acids of SEQ. ID. No. 2.

7. The nucleic acid sequence of claim 6, which encodes a peptide having an amino acid sequence which has at least 99% homology with the amino acids of SEQ. ID. No. 2.

8. The nucleic acid sequence of claim 7, which encodes a peptide consisting of the amino acid sequence of SEQ. ID. No. 2.

9. The nucleic acid sequence of claim 1, which encodes a peptide having an amino acid sequence which has at least 90% homology with the amino acids of SEQ. ID. No. 3.

10. The nucleic acid sequence of claim 9, which encodes a peptide having an amino acid sequence which has at least 99% homology with the amino acids of SEQ. ID. No. 3.

11. The nucleic acid sequence of claim 10, which encodes a peptide consisting of the amino acid sequence of SEQ. ID. No. 3.

12. The nucleic acid sequence of claim 1, which encodes a peptide having an amino acid sequence which has at least 90% homology with the amino acids of SEQ. ID. No. 4.

13. The nucleic acid sequence of claim 12, which encodes a peptide having an amino acid sequence which has at least 99% homology with the amino acids of SEQ. ID. No. 4.

14. The nucleic acid sequence of claim 13, which encodes a peptide consisting of the amino acid sequence of SEQ. ID. No. 4.

15. A peptide encoded by the nucleic acid sequence of claim 1, wherein said peptide is conjugated to polyethylene glycol.

16. A method for treating osteoporosis, Pagets disease or Prolactinoma, comprising administering to a patient a peptide encoded by the nucleic acid sequence of claim 1.

17. The method of claim 16, wherein said peptide is administered to said patient as an intramuscular injection or a nasal aerosol.

18. The method of claim 16, wherein said patient is a human being.
